(19) United States
(12) Patent Application Publication
(10) Pub. No.: US 2010/0261230 A1
(43) Pub. Date: Oct. 14, 2010

(54) SYSTEM COMPRISING DUAL-SIDED THERMAL CYCLER AND EMULSION PCR IN POUCH

(75) Inventors: David LIU, Los Altos, CA (US); Joon Mo YANG, Redwood City, CA (US); Maryam SHARIATI, Sunnyvale, CA (US); Sam WOO, Redwood City, CA (US); David M. COX, Foster City, CA (US); Dennis A. LEHTO, Santa Clara, CA (US)

Correspondence Address: KILYK & BOWERSOX, P.L.L.C., 3925 CHAIN BRIDGE ROAD, SUITE D401, FAIRFAX, VA, 22030, US

(73) Assignee: APPLIED BIOSYSTEMS, LLC, Carlsbad, CA (US)

(21) Appl. No.: 12/756,783

(22) Filed: Apr. 8, 2010

Related U.S. Application Data
(60) Provisional application No. 61/307,428, filed on Feb. 23, 2010.
(60) Provisional application No. 61/167,781, filed on Apr. 8, 2009.
(60) Provisional application No. 61/167,766, filed on Apr. 8, 2009.

(51) Int. Cl. C12P 19/34; C12M 1/00

(52) U.S. Cl. 435/91.2; 435/289.1

(57) ABSTRACT

A system and method are provided for large volume sample amplification adaptable for use with conventional PCR-based reactions as well as emulsion-based PCR reactions. A sample is retained in a pouch or flexible bag which permits bulk PCR amplification with efficient heat-transfer properties. For applications involving emulsion-based PCR amplification, the system and method provide improved uniformity in emulsion amplification and can be used to amplify large or small volume emulsions rapidly and reproducibly.

### CROSS-REFERENCE TO RELATED APPLICATIONS

The present application claims the benefit of the earlier filing date of U.S. Provisional Patent Applications Nos. 61/307,428, filed Feb. 23, 2010, 61/167,781, filed Apr. 8, 2009, and 61/167,766, filed Apr. 8, 2009, each of which is incorporated herein in its entirety by reference.

### FIELD

The present teachings relate to devices, systems, and methods for preparing and reacting within emulsions, including emulsions useful in biological reaction processes, such as, for example, amplification processes. The present teachings also relate to devices, systems, and methods for conducting PCR reactions including those associated with PCR amplification of emulsions.

### INTRODUCTION

A number of biological sample analysis methods rely on sample preparation steps as a precursor to carrying out the analysis methods. For example, a precursor to performing many biological sequencing techniques (e.g., sequencing of nucleic acid) includes amplification of nucleic acid templates in order to obtain a large number of copies (e.g., millions of copies) of the same template.

Polymerase chain reaction is a well understood technique for amplifying nucleic acids which is routinely used to generate sufficiently large DNA populations suitable for downstream analysis. Recently, PCR-based methods have been adapted to amplifying samples contained within emulsions for sequencing applications. In such amplification methods a plurality of biological samples (e.g. nucleic acid samples) may be individually encapsulated in microcapsules of an emulsion and PCR amplification conducted on each of the plurality of encapsulated nucleic acid samples simultaneously. Such microcapsules are often referred to as “microreactors” since the amplification reaction occurs within the microcapsule.

In some cases, the microcapsule may include an enrichment bead and the amplification process may be referred to as bead-based emulsion amplification. In such a technique, beads along with DNA templates are suspended in an aqueous reaction mixture and then encapsulated in a water-in-oil emulsion. The template DNA may be either bound to the bead prior to emulsification or may be included in solution in the amplification reaction mixture. For further details regarding techniques for bead emulsion amplification, reference is made to PCT publication WO 2005/073410 A2, entitled “NUCLEIC ACID AMPLIFICATION WITH CONTINUOUS FLOW EMULSION,” which published internationally on Aug. 11, 2005, and is incorporated by reference in its entirety herein.

According to various methodologies, performing bead-based emulsion amplification relies on the formation of an emulsion which encapsulates a template DNA strand, a bead upon which DNA strands amplified from the template DNA strand are retained and a reagent mixture for supporting the amplification reaction. As noted above, the emulsion typically comprises a water-in-oil emulsion with the aqueous phase (e.g., dispersed phase) including the reagent mixture and the beads, and the continuous phase including oil or other non-aqueous liquid partially or completely immiscible in water.

A significant consideration for a sequencing workflow using emulsions relates to the amplification of DNA within individual microreactors once the emulsion has been formed. A typical emulsion preparation for a sequencing reaction may have a volume of approximately 1 ml or less. Such relatively small volumes may be retained in a standard microtube (for example with a volumetric capacity of approximately 1 ml, 1.7 ml, or 2 ml). These microtubes are of a size and dimensionality to reside within the thermal block of commercially available thermal cyclers such as the Applied Biosystems 9700 thermal cycler. Amplification of the constituents present in the emulsion by polymerase chain reaction may then be conducted according to known methods.

Various problems arise, however, where the desired emulsion volume exceeds the capacity of the microtubes used with conventional thermal cyclers. For example, for a larger volume emulsion preparation it may be necessary to prepare separate emulsions or distribute aliquots in separate microtubes to be thermal cycled independently. Consequently, additional effort and care must be taken when preparing and reacting large volume emulsions increasing the amount of time and labor involved to achieve the desired amplification. Furthermore, each portion of the subdivided emulsion may be subject to increased variability arising from the local reaction characteristics which may differ from one microtube to the next (for example due to thermal variability within the block of the thermal cycler).

It will be appreciated that the step of amplification of the emulsion through PCR (ePCR) is an important step in many next generation sequencing workflows. Oftentimes, a sample to be amplified and sequenced is relatively precious and loss or inefficient sample amplification is not acceptable. In those emulsion preparations where a relatively large emulsion volume is to be amplified the manner and apparatus in which the ePCR is conducted becomes significant.

Another aspect of ePCR reactions occurring in relatively large volumes relates to the heat-transfer characteristics of the reaction which is different from that of conventional (aqueous phase only) PCR reactions where the reagents for the conventional PCR reactions have fluidic properties similar to that of water alone. Large volume ePCR therefore should take into consideration the multiphase composition and characteristics of the fluidic constituents (e.g. aqueous and non-aqueous phases) which may possess different fluidic properties affecting the manner in which the temperature ramping of the reaction is conducted. For the reasons discussed above large volume emulsion amplification may benefit from a different engineering solution from that of the traditional smaller volume PCR-based reactions.

It is therefore desirable to provide a more convenient emulsion amplification system and method, for example, one that reduces the activity required by a user during amplification of the emulsion and/or that can be automated. It also may be desirable to provide an emulsion amplification technique that facilitates increasing the throughput of biological sample analysis processes by increasing the efficiency of sample preparation including increasing the capacity of emulsion amplification for volumes over 1 ml.

### SUMMARY

According to various embodiments of the present teachings, a method of carrying out a polymerase chain reaction is provided and comprises positioning a pouch between and in contact with opposing surfaces of a multi-sided thermal cycler. The pouch contains an emulsion that comprises a plurality of polymerase chain reaction microreactors each containing components for carrying out a polymerase chain reaction. The method can comprise thermally cycling the emulsion in the pouch, using the multi-sided thermal cycler, to cause a polymerase chain reaction in one or more of the microreactors. The multi-sided thermal cycler can comprise a dual-sided thermal cycler in some embodiments. The pouch can be in the form of a bag or another flexible container and can comprise one or more openable and closeable ports. The multi-sided thermal cycler can have an upper end and a lower end, and positioning the pouch can comprise orienting the pouch such that the port is arranged at the upper end of the multi-sided thermal cycler.

In some embodiments, methods of heat sealing a PCR emulsion in a pouch are provided, for example, prior to positioning the pouch in a multi-sided thermal cycler. The pouch can comprise a flexible bag or flexible container and the positioning can comprise squeezing the pouch. The thermal cycling can comprise operating one or more Peltier heating and cooling units. The multi-sided thermal cycler can comprise a first unit and a second unit, and each of the first unit and the second unit can independently comprise its own respective Peltier unit and its own respective fan. In some embodiments, the pouch can comprise an opening and the method can further comprise filling the pouch with an emulsion, through the opening, and then closing the opening.

According to various embodiments, a system is provided that comprises a flexible pouch containing components for carrying out a polymerase chain reaction. A thermal cycler comprising opposing first and second heat-transfer surfaces can also be provided wherein the flexible pouch is sandwiched between and in contact with the first and second heat-transfer surfaces. The first heat-transfer surface can comprise the same material as the second heat-transfer surface, and in some embodiments the first and second heat-transfer surfaces can have the same dimensions. The system can further comprise a second pouch containing components for carrying out a polymerase chain reaction, wherein the thermal cycler further comprises opposing third and fourth heat-transfer surfaces, and the second pouch is sandwiched between and in contact with the third and fourth heat-transfer surfaces. In some embodiments, the thermal cycler can comprise a first Peltier unit defining the first heat-transfer surface, and a second Peltier unit defining the second heat-transfer surface. In some embodiments, the thermal cycler can comprise a first fan for cooling the first heat-transfer surface and a second fan, separate from the first fan, for cooling the second heat transfer surface.

According to various embodiments, the system can further comprise: a first heat-transfer surface frame stabilizing the first heat-transfer surface; a second heat-transfer surface frame stabilizing the second heat-transfer surface; and a linkage linking together the first heat-transfer surface frame and the second heat-transfer surface frame. The linkage can be configured to enable at least one of the first heat-transfer surface frame and the second heat-transfer surface frame to be moved toward and away from the other. The system can further comprise a cam assembly configured to adjust the distance of a gap formed between the first heat-transfer surface and the second heat-transfer surface when the first heat-transfer surface frame and the second heat-transfer surface frame are operationally positioned for thermal cycling. The system can further comprise a housing, wherein the first heat-transfer surface frame is fixed to the housing and the second heat-transfer surface frame is moveable relative to the housing.

According to various embodiments a system is provided that comprises an emulsifier module, an amplifier module, and an enrichment or enricher module, which together can be used to form templated beads useful in a bead-based DNA sequencing platform. In some embodiments, the system can comprise in-line filters to non-magnetically concentrate beads and perform buffer exchanges. In some embodiments, a dia-filtration unit and method can be used in lieu of a manual glycerol cushion and centrifugation. In some embodiments, beads are de-aggregated using sheer flow through a syringe valve.

According to various embodiments, the emulsion can be formed by mixing together an aqueous phase solution, a plurality of template beads, a library of templates from a sample, DNA polymerase, and a pair of primers, to form a mixture. The mixture can then be contacted with an oil phase and then emulsified to form an emulsion comprising a plurality of microreactors. For example, the emulsion can be formed as described, for example, in concurrently filed U.S. patent application Ser. No. ______ to Lau et al., entitled “System and Method for Preparing and Using Bulk Emulsion,” Attorney Docket No. 5010-480-01, which is incorporated herein in its entirety by reference.

According to various embodiments, the method can comprise enriching the templated beads using an enriching system and method as described, for example, in concurrently filed U.S. patent application Ser. No. ______ to Karger et al., entitled “Column Enrichment of PCR Beads Comprising Tethered Amplicons,” Attorney Docket No. 5010-480-03, which is also incorporated herein in its entirety by reference.

### BRIEF DESCRIPTION OF THE DRAWINGS

The accompanying drawings, which are incorporated in and constitute a part of this specification, illustrate exemplary embodiments and together with the description, serve to explain various principles. The skilled artisan will understand that the drawings, described below, are for illustration purposes only. The drawings are not intended to limit the scope of the present teachings in any way.

FIG. 1 shows a templated bead workflow from emulsion generation to bead enrichment, according to various embodiments of the present teachings.

FIG. 2 is a flowchart showing exemplary process steps that can be carried out by a method and system according to various embodiments of the present teachings.

FIG. 3 illustrates an embodiment of a reaction chamber that can be used in connection with large volume PCR-based reactions according to various embodiments of the present teachings.

FIG. 4 illustrates an exemplary configuration where multiple sample chambers are thermal cycled in parallel, according to various embodiments of the present teachings.

FIG. 5 is a cross-sectional view of a pouch embodiment comprising a bubble wrap layer and an aluminum foil layer, according to various embodiments of the present teachings.

FIGS. 6A-6F are various views of a dual-sided thermal cycling apparatus according to various embodiments of the present teachings.

FIG. 7 is a front view of a flexible pouch configured for use in containing an emulsion for an ePCR amplification reaction according to various embodiments of the present teachings.

FIG. 8 is a front perspective view of a pouch according to various embodiments of the present teachings.

FIG. 9A is a perspective view of an amplifier module having an open door and showing two heater blocks and two aprons.

FIG. 9B is an enlarged view of a portion of FIG. 9A.

FIG. 10A is a front perspective view of an amplifier module having an open door and a door-mounted fan unit, according to various embodiments of the present teachings.

FIG. 10B is an inside view of an amplifier module showing a fan unit for an interior heating unit, according to various embodiments of the present teachings.

### DESCRIPTION

According to various embodiments of the present teachings, an emulsion is created that comprises droplets of an aqueous phase, or microreactors, in which clonal amplification takes place. Microreactors containing a single template bead and a single template, called monoclonal microreactors, are desired and can be formed according to the present teachings. Some microreactors, however, can be polyclonal such that they contain multiple templates, non-clonal such that they contain no template, or multi-bead-containing, and some microreactors exhibit a combination of these features.

After the emulsion is created, it can be thermally cycled to produce, for example, more than 30,000 copies of template amplified on to each template bead. Each template bead can comprise a respective primer, for example, a P1 primer, attached to a bead. In non-clonal microreactors, the template bead cannot amplify. Although beads are referred to often herein, it is to be understood that other template or target supports can be used, for example, particles, granules, rods, spheres, shells, combinations thereof, and the like. Furthermore, although the microreactors are described herein as containing components for PCR, it is to be understood that the microreactors can contain components for reactions other than PCR, for example, components for an isothermal reactions, components for another amplification reaction, components for an enzymatic reaction, components for a ligation reaction, or the like.

After emulsion PCR is complete, some of the template beads comprise amplicons of the template formed thereon, and are herein referred to as templated beads. Templated beads comprise template beads on which amplification took place in the respective microreactors. Some of the template beads do not comprise amplicons of the template formed thereon, and are herein referred to as non-templated beads. Non-templated beads comprise template beads on which no amplification took place in the respective microreactors. The non-templated beads can also be referred to as non-amplifying beads.

The emulsion can then be broken, for example, with 2-butanol, and the templated beads and non-templated beads can be recovered and washed. Enrichment can be performed to isolate template beads from non-templated beads. In some embodiments, an enrichment bead comprising a single-stranded P2 adaptor or P2 primer can be used to capture the templated beads. The mixture of enrichment beads, enrichment bead-templated bead complexes, and non-templated beads, can then be subject to filtration followed by elution to isolate the templated beads.

In some embodiments, each of the templated beads and each of the non-templated beads can have a diameter of from 0.25 μm to 2.0 μm, from 0.5 μm to 1.0 μm, from 0.9 μm to 1.2 μm, or from 0.7 μm to 1.1 μm. In some embodiments, the one or more enrichment beads can each have a diameter, or collectively an average diameter, of from 3.0 μm to 20 μm, for example, from 5.0 μm to 15 μm, from 6.0 μm to 10 μm, or from 6.4 μm to 6.8 μm.

Reference will now be made in detail to various exemplary embodiments, examples of which are illustrated in the accompanying drawings. Wherever possible, the same reference numbers will be used throughout the drawings to refer to the same or like parts.

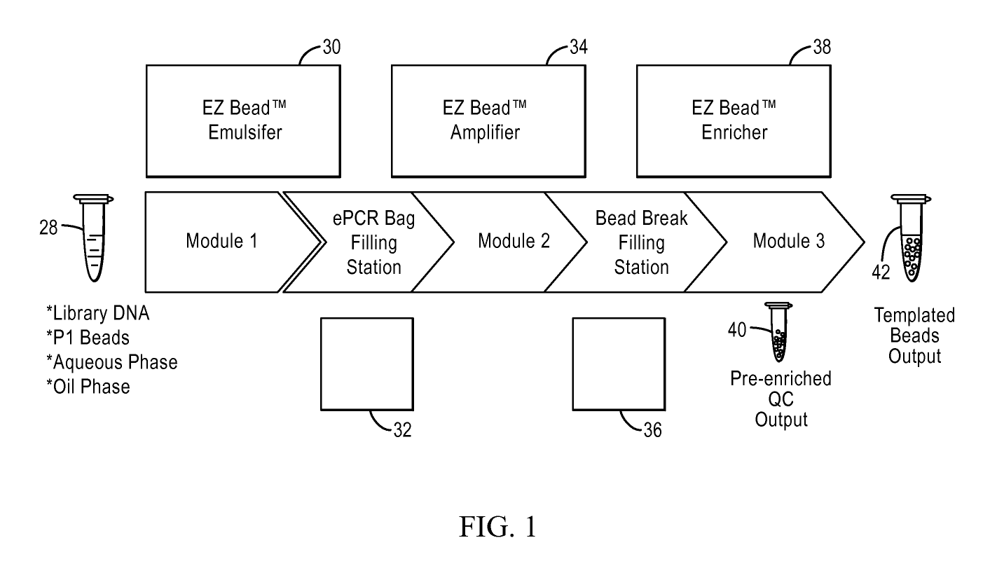

FIG. 1 shows a templated bead workflow from emulsion generation to bead enrichment, according to various embodiments of the present teachings. FIG. 1 shows an exemplary process workflow and the system components for carrying out the process. An input sample 28 to be processed by the system can comprise an aqueous phase component such as a master mix, an oil phase component such as a master mix, template beads, and a collection or library of templates such as DNA sample molecules from the same or from different samples. The aqueous phase master mix can comprise water, dNTPs, buffers, salt, and DNA polymerase. The various components for the emulsion can be brought together and emulsified in an emulsifier module 30 during a first step of the multi-step process depicted. Emulsifier module 30 is also referred to herein as module 1 in the process flow diagram shown in FIG. 1. The emulsion can be made by conventional techniques in some embodiments. After forming an emulsion using emulsifier module 30, the mixture can be poured into a pouch using an ePCR pouch filling station 32. The pouch can comprise a bag or other flexible container. After filling, the pouch can be closed or sealed, for example, by heat-sealing. The pouch and its contents can then be thermally cycled using an amplification module 34 that is also referred to herein as module 2 in the process flow diagram shown in FIG. 1. The amplification module can serve to amplify template molecules, for example, by thermal cycling. Following amplification sing amplification module 34, the contents of the pouch can be poured into a break vessel at a break filling station 36 to carry out a fourth step of the multi-step process.

After breaking the emulsion to release the beads, the templated beads can be enriched using an enrichment module 38 that is also referred to herein as module 3 in the process flow diagram shown in FIG. 1. The beads can comprise productive beads, referred to herein as templated beads. Templated beads can comprise beads that have undergone a desired reaction, for example, upon the surface of which multiple reactions have taken place. The beads can also comprise non-templated beads, which were not productive.

According to various embodiments, there can be two or more outputs of the system, including, for example, a first output 40 that includes a pre-enriched quality control output that can provide a user with information on bead clonality, for example, yield, purity, concentration, and the like. A second output 42 can be provided that includes templated beads that are ready for further processing such as terminal transferase modification, deposition on a slide or in a flow cell, a combination thereof, or the like.

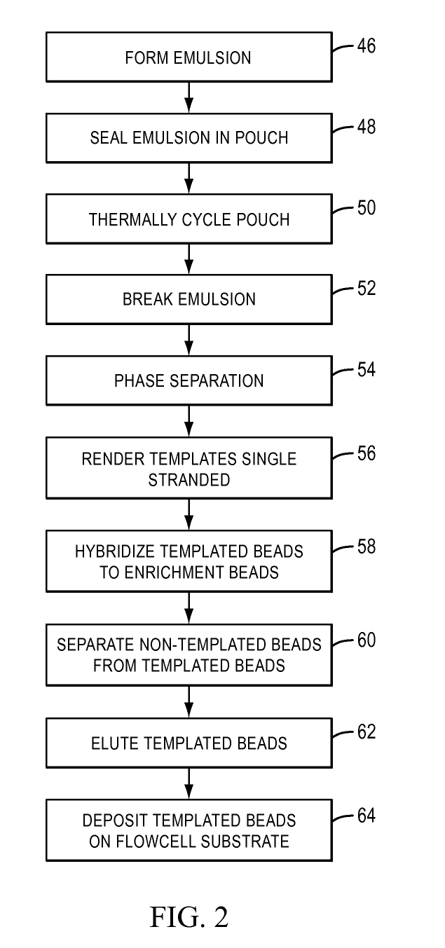

While the system described in connection with FIG. 1 comprises various different modules and stations, and process steps, it is to be understood that the system can comprise less or more modules and/or stations and that various modules and/or stations can be combined together. Furthermore, it is to be understood that the method can comprise fewer or more steps than the exemplary steps described in connection with FIG. 1 can each independently be omitted or combined with one or more other steps. In some embodiments, other amplification reactions, isothermal amplification reactions, enzymatic reactions, biological reactions, and the like, can be carried out instead of or in addition to a polymerase chain reaction. Moreover, additional steps can be provided in the method as exemplified with reference to FIG. 2.

FIG. 2 is a process flow diagram showing various process steps associated with a method according to various embodiments of the present teachings. As with FIG. 1, the process steps shown in FIG. 2 can each independently be omitted, substituted, or combined with one or more other process steps. As shown in FIG. 2, a first step 46 of the method can comprise forming an emulsion. The emulsion can be formed according to any of the various embodiments of the present teachings and as described herein. In a next step 48, the emulsion is sealed in a pouch. In an exemplary embodiment, the pouch can comprise a heat-sealable material and the sealing can comprise heat sealing the emulsion in the pouch. The sealed pouch can then be thermally cycled as depicted by process step 50.

In an exemplary embodiment, a dual-sided thermal cycler is used to amplify the emulsion in the pouch. The amplification can result in templated beads each comprising amplicons of a respective template tethered or hybridized to a primer pre-deposited on a surface of a respective template bead. The method can further comprise an emulsion breaking step 52 followed by a phase separation step 54, tailored to separate and/or purify the templated beads from the remainder of the emulsion. A denaturing step 56 can be provided to render the templates tethered to the templated beads, single stranded.

Templated beads bearing the single-stranded templates can be hybridized to enrichment beads to form a capture complex, as depicted at step 58 and described in more detail below. In the next step, the templated beads captured in the capture complexes can be separated from non-templated beads in a separation step 60, for example, using a size-exclusion technique. In a next step 62, the productive or templated beads are separated or eluted from the capture complexes and are collected. Subsequently, the collected productive or templated beads can be made ready for other operations including, for example, deposition on a flow cell substrate or otherwise formed into an array in a flow cell.

According to various embodiments, the emulsion can be formed by mixing together an aqueous phase solution, a plurality of template-capturing beads, a collection or library of sample templates or nucleic acid fragments, DNA polymerase, other enzymes, buffers, salts, and a pair of primers, to form a mixture. The mixture can then be contacted with an oil phase and emulsified to form an emulsion comprising a plurality of microreactors. On exemplary approach to emulsification is described, for example, in concurrently filed U.S. patent application Ser. No. ______ to Lau et al., entitled “System and Method for Preparing and Using Bulk Emulsion,” Attorney Docket No. 5010-480-01, which is incorporated herein in its entirety by reference.

According to various embodiments, the emulsion can comprise an aqueous phase and an oil phase wherein the aqueous phase comprises components useful for a desired reaction, for example, components for amplifying DNA templates such as a library of templates from a single sample. In some embodiments, the emulsion comprises clonal or monoclonal reactors or microreactors containing a single DNA template molecule. Some sequencing platforms, for example, the SOLID sequencing system by Applied Biosystems, Foster City, Calif., utilize emulsion polymerase chain reaction (ePCR) approaches that provide compartmentalization of PCR reactions in discrete aqueous droplets of an inverse emulsion such as a water-in-oil (W/O) emulsion. In some embodiments, a template bead, approximately 1 μm in diameter, and comprising surface-immobilized oligo nucleotides, can be entrapped in each discrete aqueous droplet microreactor. Each microreactor can also contain PCR reagents such as a forward primer, a reverse primer, a DNA polymerase, and a single DNA sample molecule.

In some cases, some of the microreactors can comprise some of the components but not others. For example, some microreactors can contain no template and no DNA polymerase, and would not be expected to yield a templated bead. According to various embodiments, the microreactors can contain other components for reactions other than PCR, for example, components for an isothermal amplification, components for another amplification reaction, components for an enzymatic reaction, components for a ligation reaction, or the like.

In various embodiments, the emulsion is thermally cycled from approximately 64° C. to 96° C. for 40 or 60 cycles (depending on the length of the template molecule being used). Subjecting the microreactors to PCR conditions in this manner results in clonal amplification yielding a product that is composed of a singular DNA species. The amplification conditions can cause a templated bead to be formed in many of the microreactors. Concentrations of components can be used to minimize the number of microreactors containing two or more templated beads. The microreactors can include microreactors that contain no template molecule or no template bead and thus do not produce a templated bead.

The emulsion preparation system and method can be adapted to readily prepare a wide range of different emulsion volumes, for example, of from approximately 5 mL to 250 mL or more, without maintaining a stock of differently sized or configured consumables to accommodate a particular emulsion volume. The emulsion exhibit small drop size variation, a slow rate of reversion or phase separation, and an adaptability to a wide variety of volume sizes. Additionally, the emulsion preparation apparatus of the present teachings is cost-effective, user-friendly, and robust, and provides a reproducible means to prepare inverse emulsions for ePCR.

In some embodiments, the present teachings provide devices, methods, and formulations for the preparation of inverse (water-in-oil) emulsions for polymerase chain reactions. In various embodiments, the discrete aqueous phase (droplets) can entrap a particle, for example, a magnetic particle of about 1 μm diameter size and having oligonucleotides such as one or more different types of primers immobilized on its surface. The discrete aqueous phase droplet can also comprise PCR reagents such as dNTPs, enzymes, co-enzymes, salts, buffers, surfactants, and a template molecule such as a DNA sample. The template molecule can be a sample DNA molecule, for example, a template from a library of templates from a single sample. The continuous phase can comprise oil with or without added surfactants that have hydrophilic-lipophilic-balances (HLB) values equal to or less than 5.0 and below. According to various embodiments of the invention, the surfactants can comprise a mixture of surfactants having various HLB values. Those who are skilled in the art can appreciate that the surfactant affinity different (SAD) of an oil phase can be adjusted by using various surfactants with various HLB values such that a stable inverse (water-in-oil) emulsion can be prepared.

The liquid oil phase can comprise a mineral oil such as Petroleum Special, an alkane such as heptadecane, a halogenated alkane such as bromohexadecane, an alkylarene, a halogenated alkyarene, an ether, or an ester having a boiling temperature above 100° C. The oil phase can be insoluble or slightly soluble in water. The ratio between the continuous oil phase and the discrete aqueous phase may range from 1/0.1 v/v to 4/1 v/v, from 0.5/1 to 3/1, from 0.8/1 to 1/1, or as desired.

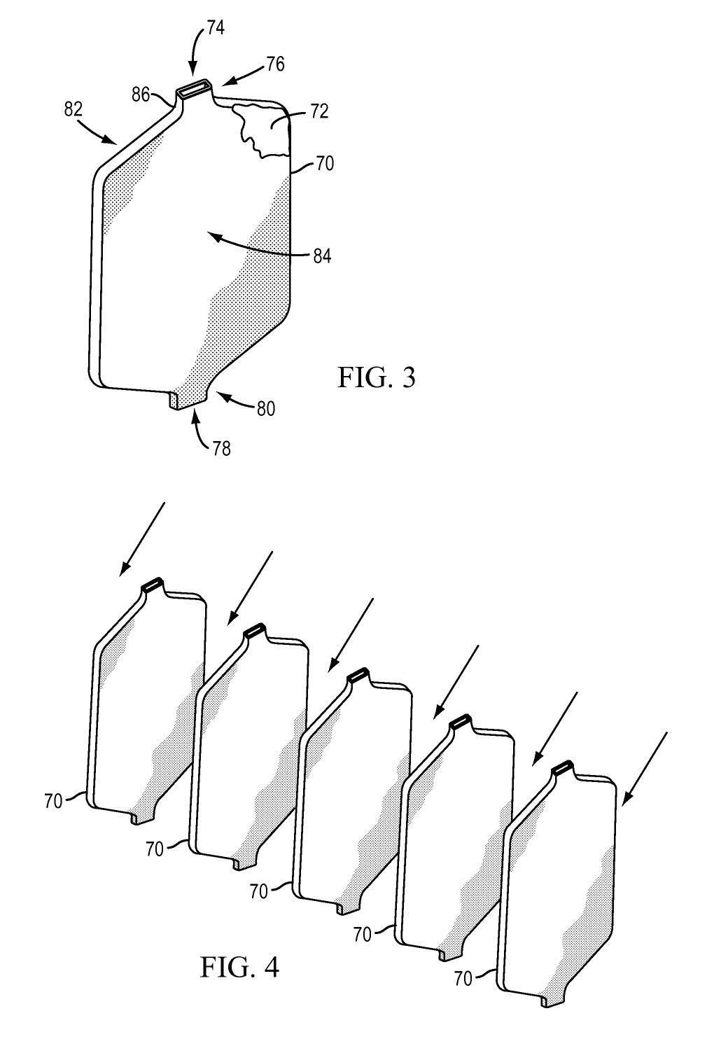

FIG. 3 illustrates an embodiment of a sample chamber that can be used in connection with large volume PCR-based reactions including emulsion PCR (ePCR) reactions for sequencing applications. As shown, pouch 70 defines a reaction chamber 72. Pouch 70 can be fabricated as a relatively thin-walled film or material. Pouch 70 can comprise one or more walls comprising a metal, a plastic, a polymer, a combination thereof, and the like. In some embodiments, pouch 70 comprises aluminum, polypropylene, or a combination thereof. Reaction chamber 72 can be configured to contain a desired liquid volume, for example, from 2 ml to 200 ml, from 5 ml to 150 ml, or more. The walls of pouch 70 can be, for example, less than approximately 1.0 mm, less than 0.9 mm, less than 0.7 mm, or less than 0.5 mm in thickness, to enhance heat-transfer.

Reaction chamber 72 can have an inlet port 74, flow through which can be controlled by an inlet valve 76. Reaction chamber 72 can have an outlet port 78, flow through which can be controlled by an outlet valve 80.

In a template amplifying procedure, for example, in a PCR thermal cycling procedure, pouch 70 can be arranged and in contact with a first heating and cooling unit positioned on a first side 82 of pouch 74, and a second heating and cooling unit comprising a heat-transfer surface in contact with a side 84 of pouch 70. In some embodiments, the heating and cooling units can each comprise a Peltier unit. As shown in FIG. 3, when oriented as shown in perspective, air bubbles that might be present in reaction chamber 72 can move to the narrowed top 86 of pouch 70 and out of the way, thus prevented from causing thermal deviations and/or cool spots in the emulsion during thermal cycling.

The pouch can be squeezed together by the two opposing thermal plates such that the pouch exhibits a uniform thickness across its entirety.

Sample constituents and reagents (including emulsions) can be introduced into reaction chamber 72 via inlet port 74 that provides a convenient manner in which to fill pouch 70. Outlet port 78 can enable convenient dispensing of reaction product and constituents from pouch 70. One or both of inlet port 74 and outlet port 78 can serve as a vent for reaction chamber 72 to facilitate filling or dispensing. Each of inlet port 74 and outlet port 78 can be configured with its respective valve to selectively control the flow of fluid in and out of reaction chamber 72 and to isolate the reaction contents during thermal cycling.

For ePCR-based reactions, the material composition of the reaction chamber can be different from that used in connection with standard PCR reactions as a result of the reactant mixture being confined within a non-aqueous component, for example, oil. The oil can prevent the reactant mixture from interacting with the walls of the reaction chamber. In general, the material used for constructing the reaction chamber may be selected so as to impart desirable heat-transfer properties which permit the efficient heating of the sample contained within. In some embodiments, the reaction chamber can comprise a material that minimizes undesirable chemical interaction between the reaction chamber material itself and components of the reaction, for example, the chamber material can comprise a polymer coated aluminum foil material.

In various embodiments, the sample chamber can be “sandwiched” within a device capable of efficiently heating and cooling the sample chamber according to temperatures and timing associated with PCR-based reactions. For example, the sample chamber can be contacted by or otherwise in heat-transfer communication with opposing Peltier-based heating and cooling units. One or more Peltier-based devices can be used, for example, a single unit comprising a dual-sided thermal cycler configured to heat both sides of the sample chamber simultaneously. In some embodiments, two separate Peltier-based units are used one for each side of the sample chamber.

In some embodiments, the sample chamber can be oriented vertically during PCR or ePCR amplification preparation. For example, an ePCR reaction workflow can call for the introduction of an emulsion through the inlet port of the device. The emulsion can be introduced, for example, by pumping, pipetting, syringe, gravity, suction, vacuum, or any other suitable method.

Thermal cycling conducted using the two-sided heating and cooling method as described herein can provide more efficient heat-transfer in comparison to heating and cooling only one side of the sample chamber. Single-sided heat-transfer units, such as those used by placing the sample chamber on a conventional PCR thermal cycler with a flat block, can result in slower heat-transfer and thermal uniformity in the sample reaction constituents. The present dual-sided heating and cooling approach can greatly facilitate relatively large volume PCR samples, for example, of more than 100 ml. If the pouch as a variable thickness, air gaps and thermal deviations can be caused. Accordingly, the present teachings provide a uniformly spaced gap and a squeeze feature to ensure that the pouch is squeezed to conform to the gap between the two thermal cycling plates, and thus exhibits a uniform thickness across its entirety during amplifying and/or thermal cycling. A space or gap formed between two thermal cycling or heating plates can help ensure substantially uniform and complete contact between the pouch and the plates, for example, complete contact with two Peltier blocks. A spring-loaded or other biasing device can be used to squeeze the pouch without rupturing it. In some embodiments, the pouch can be provided with corners, squared edges, or the like, so that fluid or emulsion is evenly disbursed or distributed. The pouch can conform to the interior dimensions of the amplifier or thermal cycler, for example, to the gap formed therein.

In some embodiments, the sample chamber can be fabricated from a clear or partially transparent material such as a clear plastic, for example, polyethylene terephthalate, a polyolefin film, or the like. This can enable the contents of the reaction chamber to be readily visualized and can be helpful in filling and dispending sample.

FIG. 4 illustrates an exemplary configuration wherein multiple sample chambers, each in the form of a pouch 70, are thermally cycled in parallel. Such a configuration can use a multiplicity of heating and cooling units between pouches 70, with pouches 70 being sandwiched there between. The positions of the heating and cooling units (not shown) are indicated by the arrows.

According to various embodiments of the present teachings, the sample chamber can be configured as a thin-walled container or sample reaction pouch or bag. By virtue of the construction of the sample reaction pouch, there is relatively little thermal mass to block or alter heat-transfer into the sample reaction components from the heating and cooling device. Like the embodiments of the sample chamber described above, the sample reaction pouch can have an inlet port, an outlet port, or both, to facilitate introduction and removal of the reaction constituents. Furthermore, the dimensionality of the pouch can be configured to accommodate reaction volumes of various sizes. The reaction pouch can be operationally positioned on a heating and cooling block of a thermal cycler to provide heat-transfer into the reaction constituents contained by the pouch. Furthermore, a weight, downward pressure, or clamp can be used to further ensure efficient contact between the surface of the pouch and the heating and cooling block.

In certain embodiments such as those where the sample reaction pouch is to be used in connection with a conventional thermal cycler having a heated lid, it may be advantageous to provide an insulating material on at least a portion of the pouch. The insulating material can reside between the pouch and a heated lid of a thermal cycler, to insulate the reaction contents from the heat transmitted from the lid during thermal cycling. Additionally, an insulating material can be provided for thermal cycling instruments with or without a heated lid to improve the insulating properties of the pouch and to maintain a desired temperature transmitted from a heating and cooling block.

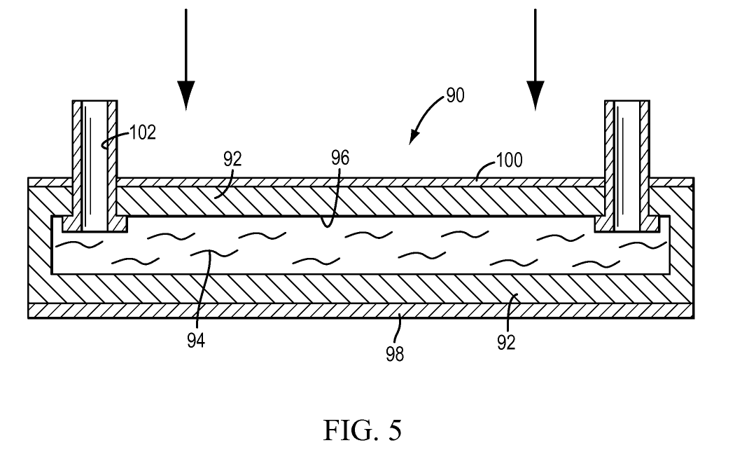

FIG. 5 is a cross-sectional view of an embodiment of a pouch 90 according to the present teachings. The thicknesses of the various materials making up the pouch are visually exaggerated and not to scale with respect to one another or with respect to the reaction chamber.

Pouch 90 comprises a reaction chamber sidewall 92 comprising, for example, a polyethylene terephthalate polymer, defining a reaction chamber 96. Sidewall 92 can comprise one or more reaction chamber side walls. Sidewall 92 can comprise one or more heat-sealed edges. In the embodiment shown, a force would be applied to pouch 90 in direction shown by the two large arrows, to force pouch 90 against a heat-transfer surface of a thermal cycler. To facilitate heat-transfer from the thermal cycler to reaction contents 94 disposed in reaction chamber 96 defined by sidewall 92, a heat-transfer layer 98 can be provided on at least one side of pouch 90. In the embodiments shown, heat-transfer layer 98 comprises an aluminum foil layer having a thickness of 1.0 mm or less, for example, a thickness of 0.8 mm or less, a thickness of 0.7 mm or less, or a thickness of 0.5 mm or less.

Pouch 90 can be provided with an insulating layer 100 that can comprise, for example, a bubble wrap material, a plastic material, an open cell material, a closed cell material, or the like. Insulating layer 100 can be used for insulating contents 94 of reaction chamber 96 from undesired heat generated by a heated lid if the thermal cycler used includes a heated lid. In some embodiments, insulating layer 100 can instead comprise an aluminum or metal layer configured to retain heat in reaction chamber 96. An ePCR emulsion can be introduced to pouch 90 through an inlet tube 102 formed through wall 92. Inlet tube 102 can be crimped shut, plugged, or otherwise closed after introduction of an emulsion to pouch 90. Pouch 90 can also be provided with an outlet tube 104 that can similarly be crimped, plugged, or otherwise closed. Tube 102 and tube 104 can be used as vents during filling or emptying pouch 90.

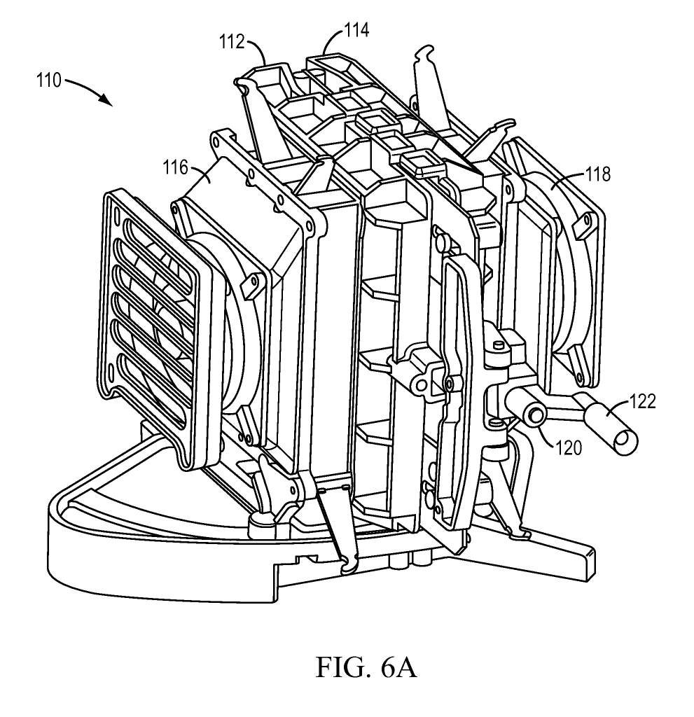

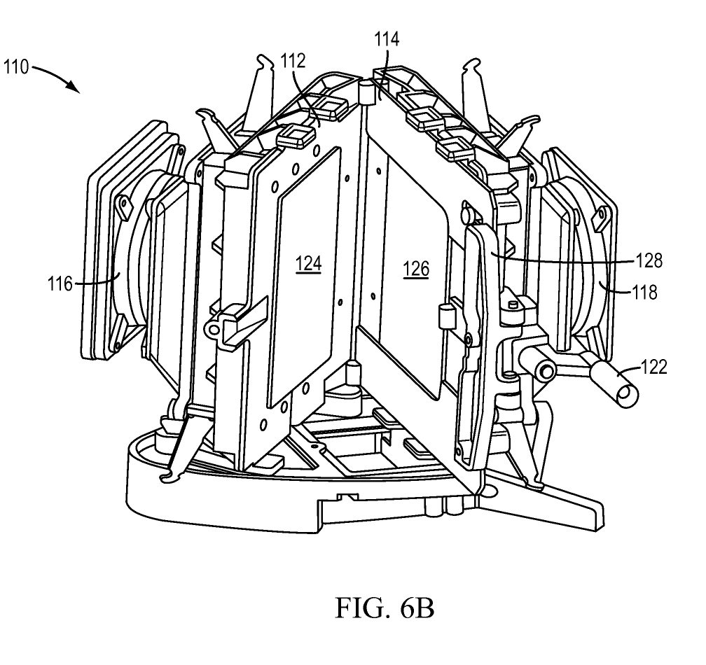

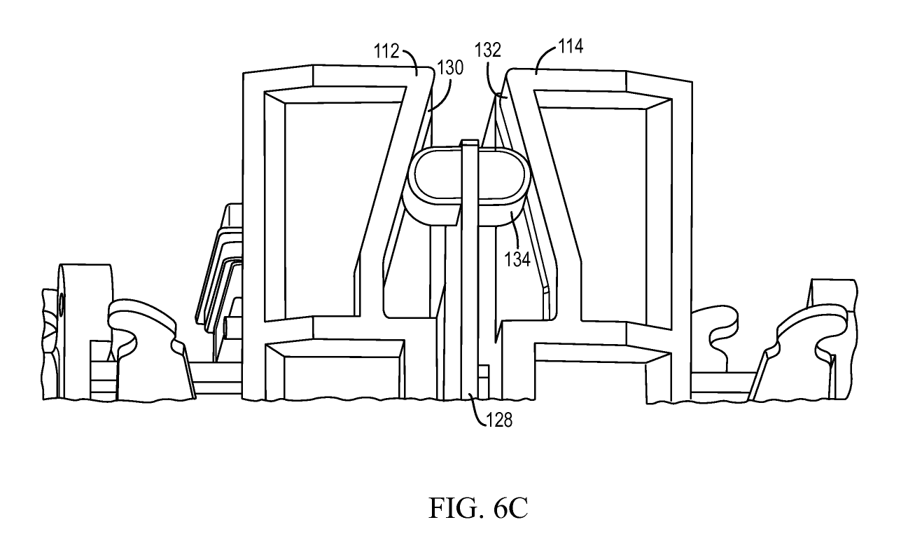

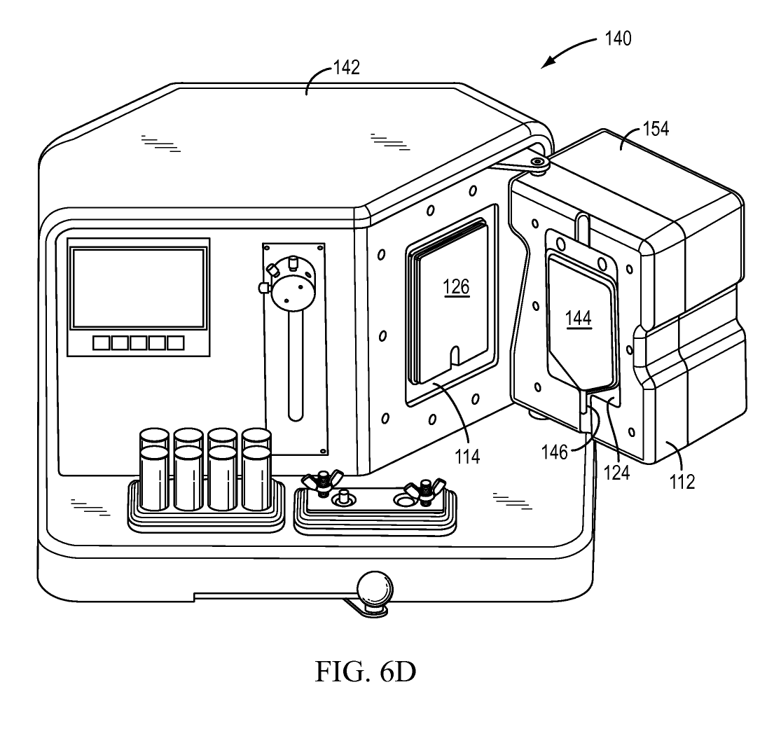

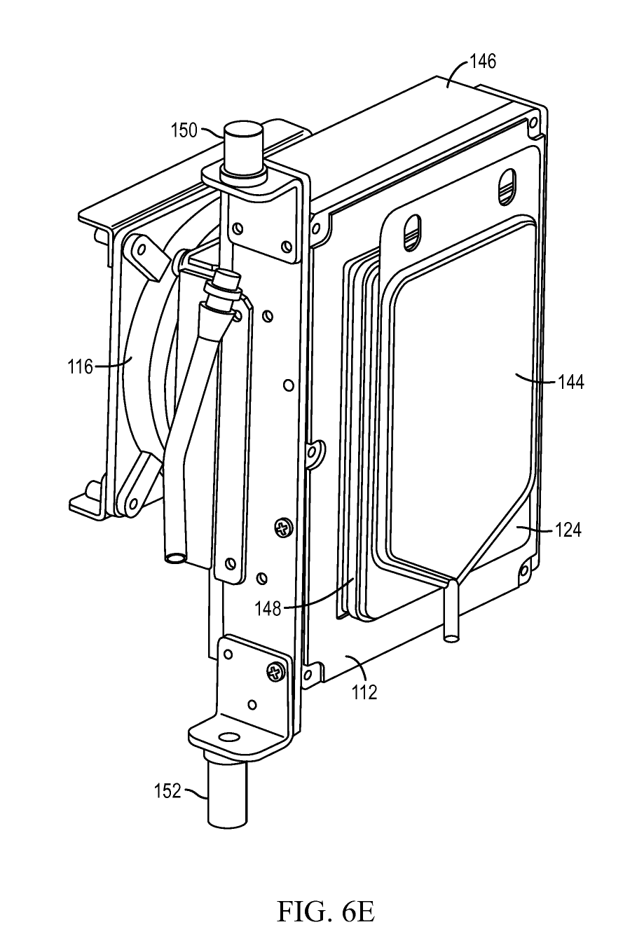

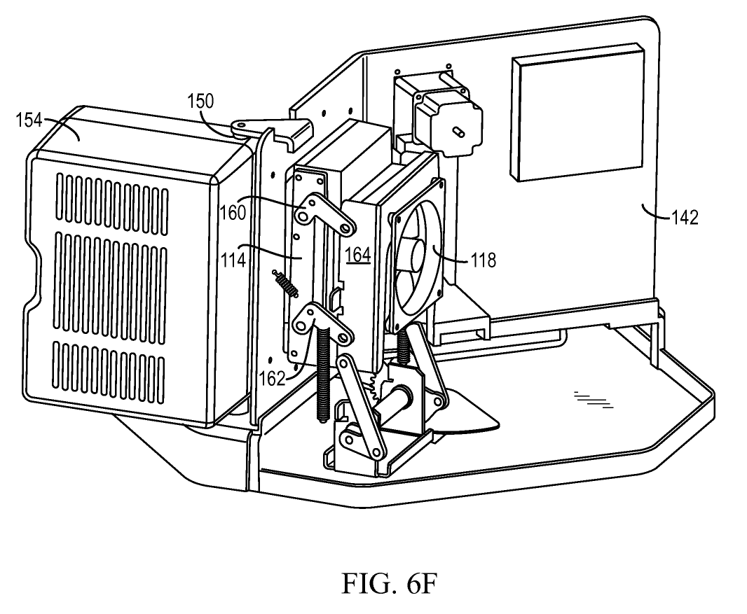

FIGS. 6A-6F illustrate various embodiments of a dual-sided thermal cycling apparatus according to the present teachings. The dual-sided thermal cycler can comprise two or more heating elements or blocks which contact opposing sides of the sample container or pouch. Peltier-based heating blocks similar to those used on conventional single block thermal cyclers can be adapted for use in dual-sided thermal cycling applications. One or more of the heater elements or blocks can be mounted to a moveable or pivoting frame. The frame pivots such that in a closed position (FIG. 6A) the heating elements or blocks engage with a pouch disposed in the system, and in an open position (FIG. 6B) the heating elements or blocks are spaced apart sufficiently to permit access to the pouch. In various embodiments, one of the heater blocks can fixed to a base while the other slides in and out relative to the first, for example, in a pivoting or displacing motion.

In various embodiments, crank or handle 122 (FIGS. 6A-6B) can be provided which moves one or more spacer blocks capable of varying the gap between the plates. The system can be capable of being adjusted manually or automatically, for example, by spring loading, to provide a suitable gap size for containment of and thermal contact with the pouch. It will be appreciated by one of skill in the art that other mechanisms by which to adjust the blocks and/or gap with respect to one another exist, as well as mechanisms to provide the opening and closing.

FIG. 6A is a perspective view of a dual-sided thermal cycling apparatus 110 according to various embodiments of the present teachings. Dual-sided thermal cycling apparatus 110 comprises a first Peltier-based heating and cooling unit 112 and a second Peltier-based heating and cooling unit 114. Heating and cooling unit 112 comprises a fan unit 116 for rapidly cooling a heat-transfer surface. Heating and cooling unit 114 comprises a fan unit 118 for rapidly cooling a respective heat-transfer surface (not shown). A clamping mechanism is provided for clamping together heating and cooling unit 112 and heating and cooling unit 114.

As shown in FIG. 6B, heating and cooling unit 112 comprises a heat-transfer surface 124. Heating and cooling unit 114 comprises a respective heat-transfer surface 126. Each of heating and cooling units 112 and 114 can comprise a Peltier heating and cooling unit. Each of heat-transfer surfaces 124 and 126 can comprise an exposed surface of a respective heater block. A gap adjustment cam assembly 128 is provided, for example, to adjust the gap between heat-transfer surface 124 and heat-transfer surface 126 when heating and cooling units 112 and 114 are brought together for thermal cycling.

In various embodiments, the frames can have ramped surfaces (FIG. 6C) which are capable of acting as cams to control the spacing between the plates or blocks. Thus the apparatus can be designed in such a way so as to accommodate a desired pouch capacity engaging with sufficient force to permit good thermal contact without crushing the pouch. In various embodiments, the plates are designed to be aligned in a generally parallel orientation so that the pouch or container resides between the plates in such a manner so as to have a uniform thickness. This configuration helps to maintain the uniformity of the heating and cooling of the contents of the pouch.

As shown in FIG. 6C, gap adjustment cam assembly 128 can comprise an adjustment member 134 that can be extended or retracted along an incline surface 130 of heating and cooling unit 112 and along an inclined surface 132 of heating and cooling unit 114. As one skilled in the art will appreciate as adjustment member 134 is moved upwardly in the view shown in FIG. 6C, heating and cooling unit 112 and 114 can be made to move away from one another, increasing the distance of the gap between heat-transfer surface 124 and heat-transfer surface 126. Conversely, as adjustment member 134 is moved downwardly in the view shown in FIG. 6C, a biasing device (not shown) can cause heating and cooling units 112 and 114 to move toward one another decreasing the distance of the gap between heat-transfer surface 124 and heat-transfer surface 126. A handle 122 can be provided, that rotates about a pivot axis 120, to move adjustment member 134. The adjustable gap can enable dual-sided thermal cycler apparatus 110 to accommodate various sized emulsion PCR pouches according to various embodiments of the present teachings. The gap can be preset for selected volumes or pouches and the plates can be configured with a degree of expansion or contraction play to allow for changes in pouch volume due to heating and cooling.

FIG. 6D illustrates a dual-sided thermal cycler apparatus with a sample pouch secured to one of the heater elements. In various embodiments, the pouch and heater element pivot as previously described to permit contact between the opposing side of the pouch and a second heating element. Additionally, the pouch can be configured as described with an access port and/or valve that engages with other components of an instrument for transferring fluid and/or emulsion into and out of the pouch. It will be appreciated that such an apparatus can provide a mechanism to automate filling, carrying out a reaction, and evacuating the pouch as part of a larger automated processing workflow.

As shown in FIG. 6D, the dual-sided thermal cycler apparatus can be a part of a system 140 comprising a housing 142 to which heating and cooling unit 114 can be fixed. As shown, heating and cooling unit 112 and its respective heat-transfer surface 124 can be movable toward and away from heating and cooling unit 114 and its respective heat-transfer surface 126. While pouch 144 is shown with a port 146 facing downwardly, it is to be understood that pouch 144 can be provided with two ports, or a single port oriented facing upwardly.

FIGS. 6E-6F show a heater block design that incorporates a fan secured to a heat sink. In various embodiments, the fan can be configured to blow substantially perpendicularly to the heat sink for efficient cooling. In this configuration, the heater block can reside on the door side in a generally fixed position and secure the pouch in a fixed orientation when the door is closed. Additionally, the heater block can be configured to have a variable displacement characteristic to move in and out to vary the gap between the plates to thereby accommodate different pouch sizes and/or provide access to the pouch as needed or desired.

FIG. 6E is a perspective view of heating and cooling unit 112 showing its respective heat-transfer surface 124 and an ePCR pouch 144 mounted against heat-transfer surface 124. Also shown in FIG. 6E is fan unit 116 configured for rapidly cooling heat-transfer surface 124, and the respective heater block of which surface 124 is a part. FIG. 6E additionally shows Peltier unit 146 in thermal contact with heater block 148, the exposed surface of which comprises heat-transfer surface 124. Pivot pins 150 and 152 are mounted to heating and cooling unit 112 to enable swiveling, pivotal movement of heating and cooling unit 112 and its associated housing 154, as shown in FIG. 6D.

FIG. 6F shows the inside of housing 142 and the internally arranged heating and cooling unit 114. FIG. 6F shows a pair of gap adjustment levers 160 and 162 and a lever linkage frame 164.

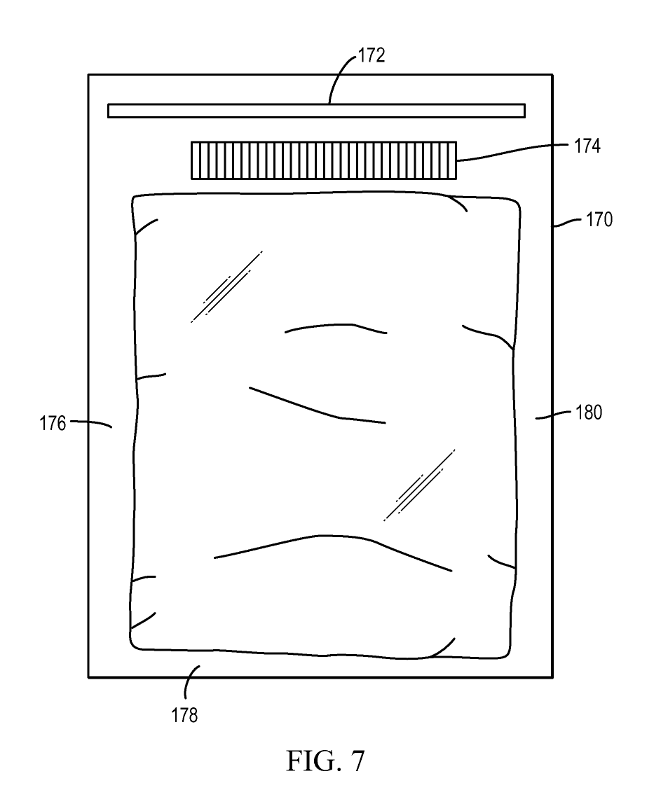

FIG. 7 illustrates a flexible pouch or bag configured for use in containing an emulsion for ePCR amplification. In various embodiments, a pouch 170 can comprise a heat resilient polymer material such as a polyester or a polypropylene capable of withstanding the temperatures associated with thermal cycling. Furthermore, pouch 170 can comprise a heat sealable material. In the configuration shown in FIG. 7, sample can be added to pouch 170 and pouch 170 can be heat-sealed to form a heat-seal 172 that prevents leakage. Air and air bubbles can be completely or substantially removed from pouch 170 prior to or during heat-sealing to form heat-seal 172. Thereafter, pouch 170 can be subjected to thermal cycling conditions to effectuate a desired PCR reaction. In some embodiments, pouch 170 can comprise three initially heat-sealed edges 176, 178, and 180, and one edge that can be heat-sealed after filling to form heat-seal 172. In various embodiments, heat-sealable pouch 170 can be configured to sequester multiple samples that are isolated from one another but that are capable of being thermal cycled within the same instrument simultaneously. Additionally, pouch 170 can incorporate an electronically readable label such as a barcode 174 or RFID element for ease of identification and automation. In some embodiments, instead of a heat seal, one or more edges of pouch 170 can be sealed before or after filling with an emulsion, by using pressure sensitive adhesive, by ultrasonic welding, by ultrasonic bonding, by sealing with a glue or adhesive, by using a melt adhesive, by using an isocyanate-based adhesive, or the like.

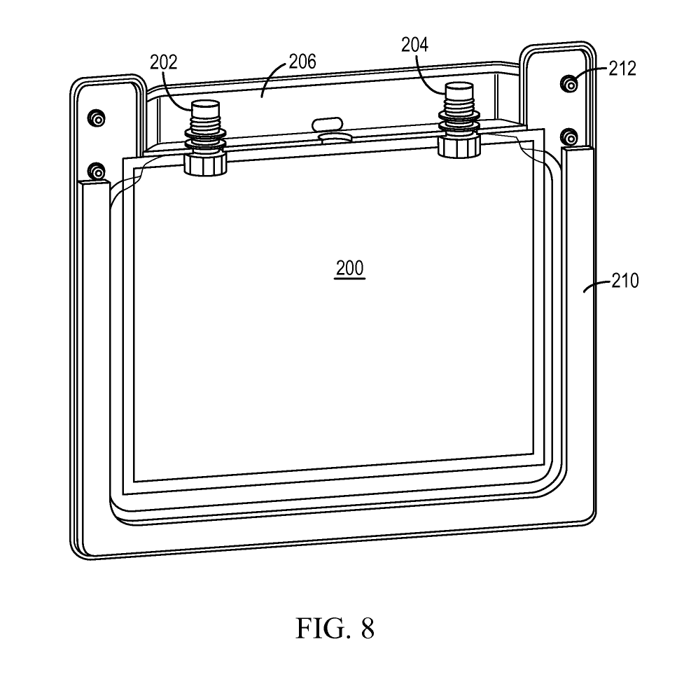

FIG. 8 is a front perspective view of a pouch according to various embodiments of the present teachings. The pouch 200 has welded fill spouts 202 and 204. Tubing welded into the bag can also be used. Fluids can be pumped in and out of pouch 200 automatically through fill spouts 202 and 204. Three smaller pouches, for example, can be used at the same time instead of one large one. The pouch can hang off of a holding frame 206 as shown. The frame can be detachable so that different specific designs for consumables can be used. Fill spouts 202 and 204 can be designed with grooves so pouch 200 can hang off of the spouts. Holding frame 206 can be injection molded, vacuum-formed, or made from bent wire. Insulating foam 210 can be provided on some or most of holding frame 206. Magnetic rivets 212 can be used, for example, to help magnetically hold holding frame 206 in place.

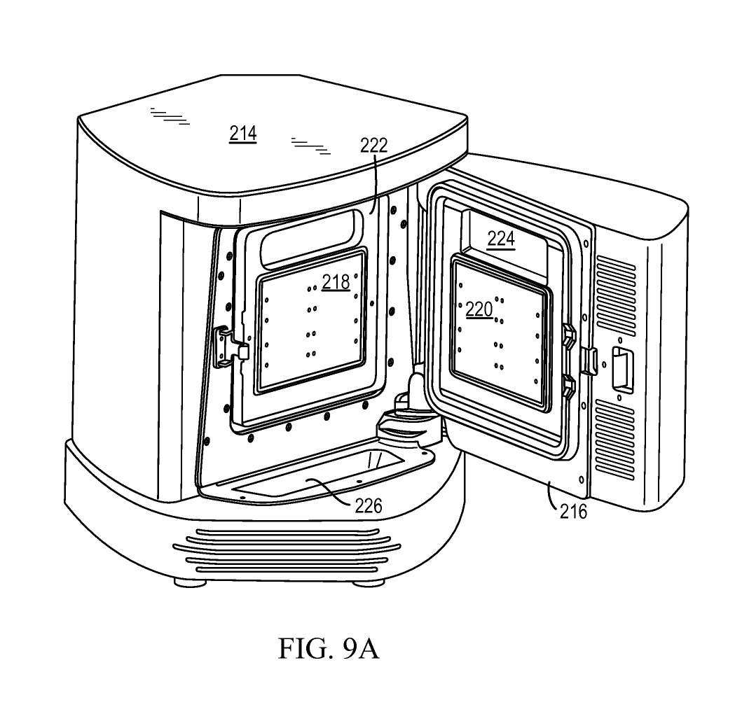

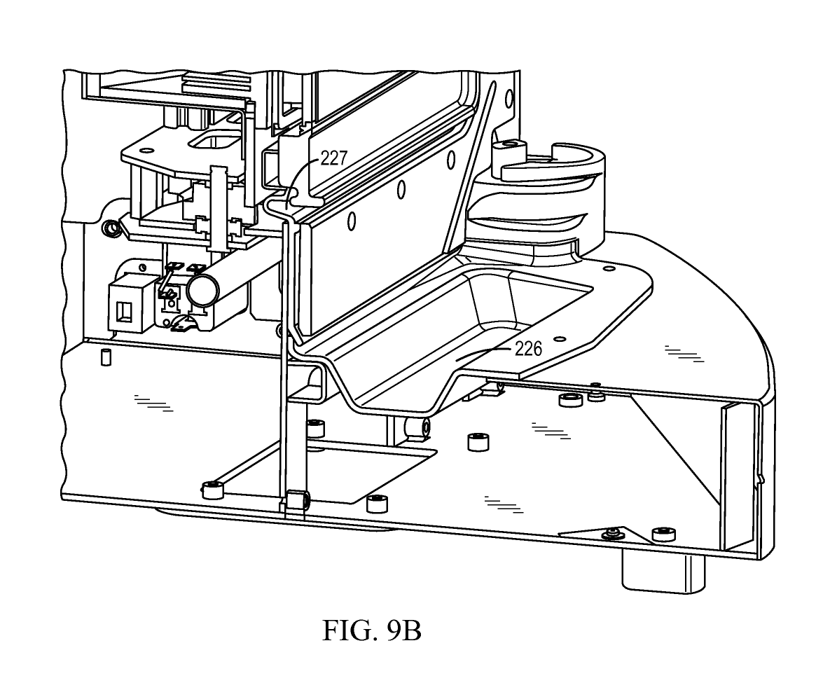

FIG. 9A is a perspective view of an amplifier module 214 having an open door 216 and showing two heater blocks 218 and 220 and two aprons 222 and 224. The heater block assembly that is mounted into the door returns to a fixed position when the door is closed. The inner heater block assembly that is in the main part of the instrument can move in and out to achieve the correct gap between the two heater blocks depending on how much liquid is in the pouch and thus how thick the pouch wants to be when it is pressed between the two plates. Because pouches can rupture and leak, the area around the pouch can be sealed to provide for secondary containment. A rolling diaphragm gasket can fill the gap between the inner heater block and the frame of the instrument. The same gasket can have an extension that ensures a sealed drip path into a drip pan 226 as shown in FIG. 9A and shown enlarged in FIG. 9B. FIG. 9B also shows an inside heater block seal 227.

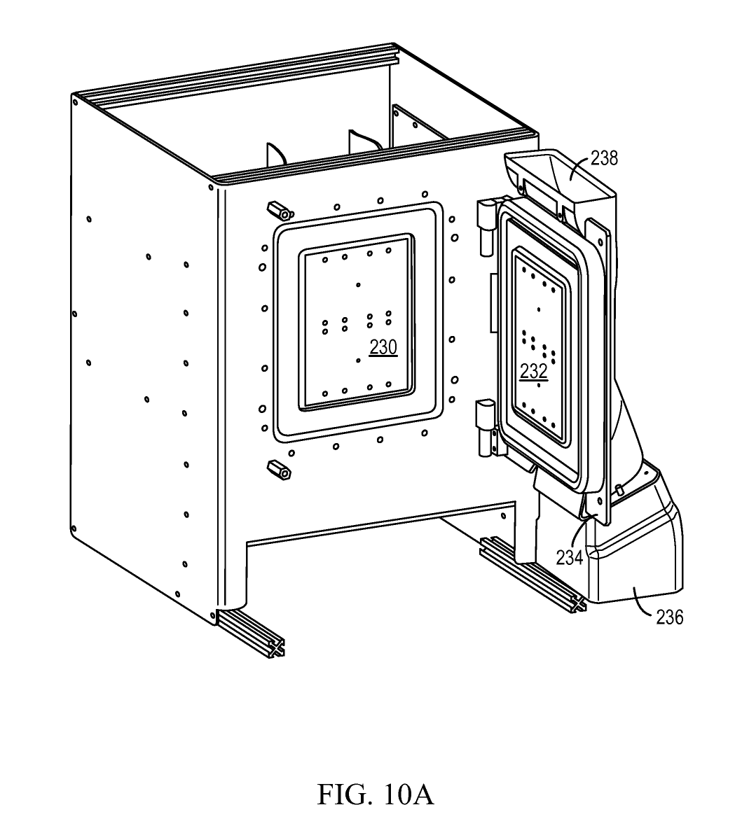

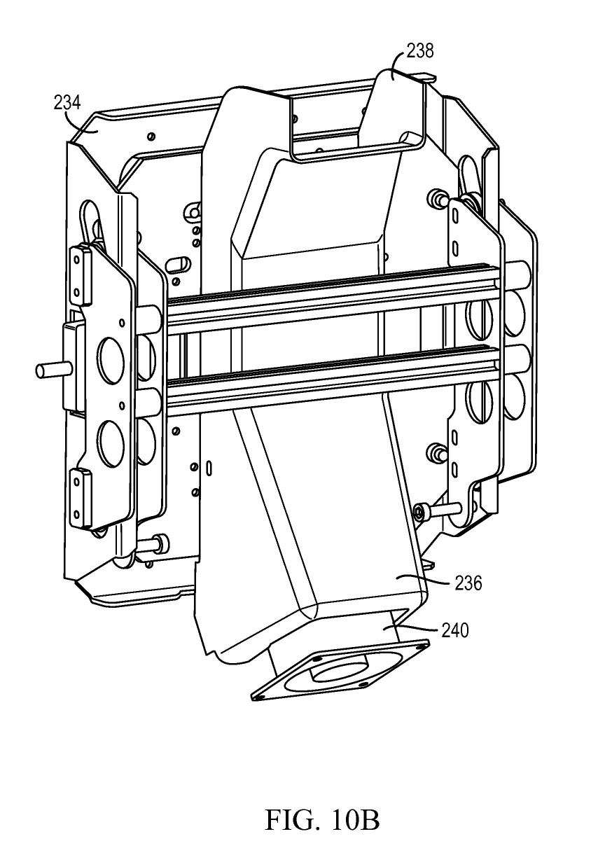

In some embodiments, the amplifier module is configured so that air is drawn in from the bottom of the front face and then exhausted from the top of the rear face. An embodiment of a cooling system for a door-mounted fan unit is shown in FIG. 10A, and an interior fan unit is shown in FIG. 10B. The heater blocks 230 and 232 can be positioned at a 45 degree angle relative to the bench top. This orientation does not require door 234 to be opened very far to access and replace a pouch (not shown). It also reduces flexing on any cable assembly running into door 234. A fan unit 236 draws air in from the main interior of the instrument and exhausts air from an exhaust port 238 into a chamber on the far side of a sheet metal partition so that hot air is not recirculated into fan unit 236. The inside heater block 230 and positioning mechanism are shown in isolation. In this position the gap between the heater blocks is at the maximum. As shown in FIG. 10B, fan unit 238 comprises a fan motor 240. While like reference numerals are used to describe FIGS. 10A and 10B, they are separate units.

In some embodiments, of the present teachings, a pouch-based thermal cycling approach is provided using a non-Peltier-based thermal cycling device. Pouches can be filled with emulsions and sealed, as described above, and placed or secured within a caddy or cage. The cage can be configured to hold one or more pouches and may further be used in connection with a conventional water bath based thermal cycler such as the Kbiosciences HYDROCYCLER.

The HYDROCYCLER is capable of thermal cycling one or more pouches using temperature controlled water baths to modulate the temperatures of the PCR reaction within the pouch. A robotic arm is used to move the cage and pouches contained therein between the water baths, achieving a rapid thermal cycling profile. It will be appreciated that the use of sealable pouches for thermal cycling in connection with such an instrument is desirable as it prevents leakage or contamination of the pouch contents while providing an efficient heat-transfer containment system.

According to various embodiments, the emulsion can be formed by mixing together an aqueous phase solution, a plurality of template beads, a collection of templates such as a library of DNA fragments from a sample, DNA polymerase, buffers, salts, dNTPs, and a pair of primers, to form a mixture. The mixture can then be contacted with an oil phase and then emulsified to form an emulsion comprising a plurality of microreactors. For example, the emulsion can be formed as described, for example, in concurrently filed U.S. patent application Ser. No. ______ to Lau et al., entitled “System and Method for Preparing and Using Bulk Emulsion,” Attorney Docket No. 5010-480-01, which is incorporated herein in its entirety by reference.

According to various embodiments, the method can comprise enriching the templated beads using an enriching system and method as described, for example, in concurrently filed U.S. patent application Ser. No. ______ to Larger et al., entitled “Column Enrichment of PCR Beads Comprising Tethered Amplicons,” Attorney Docket No. 5010-480-03, which is also incorporated herein in its entirety by reference.

Emulsion PCR (ePCR) can be used in next generation sequencing protocols such as those performed in connection with Applied Biosystems SOLiD sequencing platform. In this regard, ePCR for sequencing applications can benefit from special consideration in comparison to more traditional PCR-based amplification reactions. In conventional PCR, the volume associated with the amplification reaction is typically relatively small, on the order of between approximately 1 ul and a few hundred ul. In such instances, as previously noted above, the PCR reaction can readily be contained and performed in a microfluidic chamber or vessel such as a microtube, TLDA card, a small test tube, or a 96/384 micro-well plate. Conversely, ePCR-based reactions for sequencing applications can use a significantly larger volume reaction, for example, on the order of from 5 ml to 150 ml, or more. Consequently, conventional sample containment devices and the associated thermal cycling instruments which are used to control the temperature of the reaction, as described herein, can be used.

For large volume PCR reactions, including ePCR reactions, the heat-transfer characteristics for a large volume reaction can be significantly different from that of a conventional small volume reaction. Additionally, conventional PCR reactions have heat conduction properties which more closely resemble water as compared to ePCR reactions which contain a non-aqueous or oil-based component, significantly altering the heat-transfer characteristics of the reaction. Consequently, predicting or modeling the fluidic behavior of an ePCR reaction can benefit from not using conventional predominately aqueous based information.

Additionally, when conducting a large volume PCR or ePCR reaction it can be desirable to minimize the number of pipetting operations to reduce the amount of labor involved as well as minimize sample loss. Consequently, such amplifications reactions in which relatively large volumes are to be processed stand to benefit from the system, methods, and components of the present teachings.

In some embodiments, a sequencing system is provided that exhibits increased sequencing throughput by several orders of magnitude over gel based systems and can be instrumental in improving understanding of genomics and human disease. In some embodiments, the present teachings give end-users a most cost-effective sequencing platform.

In some embodiments, a system is provided that automates much of the workflow and greatly reduces the overall hands-on time regardless of scale. An exemplary system provides user friendliness and simplicity of operation. The system can comprise three modules, an emulsifier module, an amplifier module, and an enricher module. Each module addresses a key process in an overall workflow—emulsion preparation, emulsion thermal cycling, and bead break to templated bead enrichment, respectively.

In some embodiments, the system of the present teachings is scalable, therefore increasing the flexibility of the system to suit the throughput needs of the customer. Depending on scale chosen, the nominal bead outputs can be 125 million, 250 million and 1 billion enriched templated beads. The hands-on time for the operator can be 45 minutes or less, regardless of scale, versus a manual process for full scale which can be 145 minutes, and 340 minutes for the macro scale (8 full scale preparations). The overall time for enriched templated bead preparation can be 7-8 hours or less depending on the input library. Overall, the system of the present teachings offers great advantages in bead yield and bead purity compared to the manual templated bead process.

In some embodiments, the present teachings provide researchers with a cost-effective sequencing solution with unprecedented accuracy.

In some embodiments, an automated method of preparing templated beads is provided. In order to provide a user with maximum flexibility to meet their throughput needs, the system can be broken into at least three modules. These can include an emulsifier module, an amplifier module, and an enrichment module. Each module can be processed in a short amount of time, for example, less than 15 minutes hands-on time. The method can use plastic consumables designed to be disposable and buffers in pre-filled racks that are easy to load. The system can be automated. The system can be scale-agnostic. Even when using a challenging long mate pair template, the end-to-end performance of the automated system can be equivalent to a manual method in terms of emulsion generation, average templated bead loading, purity, and bead yields. The system exhibits minimized cross-contamination through the use of disposable plastic consumables and a cleaning protocol for non-removable hardware. The system provides users with major advantages of cost reduction, time savings, and error reduction.

In some embodiments, an emulsion can be prepared using an emulsifier module and a disposable plastic impeller. The continuous oil phase of the emulsion is a solution of emulsifiers in mineral oil, and the discrete aqueous phase (droplets) can comprise PCR components such as long mate pair template, primers, DNA polymerase, and P1 beads. The aqueous phase can be delivered using a peristaltic pump. Both the aqueous phase and oil phase can be provided as master mixes.

The emulsion can be poured into a disposable plastic thermal cycler pouch, sealed, and placed into an amplifier module. For long mate pair library templates, the emulsion mixture can be cycled for at least 40, at least 50, at least 60, or more cycles, for example, for 60 cycles. Following thermal cycling, the emulsion mixture can be poured into a disposable container and placed in an enrichment module. The emulsion can be broken with 2-butanol, washed, and enriched with an automated column-based enrichment method.

In an exemplary templated bead workflow from emulsion generation to bead enrichment, the input into the system can comprise an aqueous phase master mix (mixed with P1 beads and library DNA) and an oil phase master mix. After forming an emulsion using an emulsifier module, the mixture can be poured into a pouch using an ePCR pouch filling station. Following thermal cycling in an amplifier module, the contents can be poured into a bead break vessel and processed in the enrichment module. There can be two or more outputs of the system, including, for example, a first output that includes a pre-enriched QC output that provides a user with information on bead clonality. A second output can be provided that includes templated beads that are ready for terminal transferase modification and deposition.

In some embodiments, the system sandwiches an emulsion PCR pouch between two thermal blocks. One thermal block can be mounted on the inside of a door of the module. The pouch capacity can be, for example, 100 ml or 130 ml of emulsion, or more. The pouch can be disposable to minimize cross-contamination. The internal surfaces of the instrument can be designed to be easily wiped for cleaning.

Taken together it will be appreciated that the disclosed systems and methods of the present teachings provide an enhanced mechanism by which to conduct PCR and ePCR reactions in relatively large volumes using easy to fabricate sample chambers with improved heat-transfer characteristics.

It is to be understood that each of the publications referenced herein is independently incorporated herein in its entirety by reference.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the,” include plural referents unless expressly and unequivocally limited to one referent. As used herein, the term “include” and its grammatical variants are intended to be non-limiting, such that recitation of items in a list is not to the exclusion of other like items that can be substituted or added to the listed items.

It will be apparent to those skilled in the art that various modifications and variations can be made to the devices, systems, and methods of the present disclosure without departing from the scope its teachings. Other embodiments of the disclosure will be apparent to those skilled in the art from consideration of the specification and practice of the teachings disclosed herein. It is intended that the specification and examples be considered exemplary only.

What is claimed is:

1. A method of carrying out a reaction, comprising:
positioning a pouch between and in contact with opposing surfaces of a multi-sided thermal cycler, the pouch containing constituents for a sample reaction; and
heating the constituents in the pouch, using the multi-sided thermal cycler.

2. The method of claim 1, wherein the constituents comprise an emulsion comprising microreactors, and the heating comprises thermally cycling the emulsion to cause a polymerase chain reaction in one or more of the microreactors.

3. The method of claim 1, wherein the multi-sided thermal cycler comprises a dual-sided thermal cycler having opposing surfaces which define a gap there between which is occupied by the pouch, and the contacting comprises substantially uniformly contacting the pouch with the opposing surfaces.

4. The method of claim 1, the pouch contains an emulsion, the emulsion comprises a plurality of polymerase chain reaction microreactors each containing components for carrying out a polymerase chain reaction.

5. The method of claim 4, wherein the multisided thermal cycler has an upper end and a lower end, and the positioning comprises orienting the pouch such that the port is arranged at the upper end of the multi-sided thermal cycler.

6. The method of claim 1, further comprising heat sealing the pouch prior to the positioning.

7. The method of claim 1, wherein the pouch comprises a flexible bag and the positioning comprises squeezing the flexible bag.

8. The method of claim 1, wherein the multi-sided thermal cycler comprises one or more Peltier heating and cooling units.

9. The method of claim 1, wherein the multi-sided thermal cycler comprises a first unit and a second unit, and each of the first unit and the second unit independently comprises its own respective Peltier unit and its own respective fan.

10. The method of claim 1, wherein the pouch comprises an opening and the method further comprises filling the pouch with the emulsion, through the opening, and closing the opening.

11. A system comprising:
a flexible pouch containing components for carrying out a reaction; and
a thermal cycler comprising opposing first and second heat-transfer surfaces, wherein the flexible pouch is sandwiched between and in contact with the first and second heat-transfer surfaces.

12. The system of claim 11, wherein the flexible pouch contains components for carrying out a polymerase chain reaction.

13. The system of claim 11, wherein the first heat-transfer surface comprises the same material as the second heat-transfer surface.

14. The system of claim 11, wherein the first and second heat-transfer surfaces have the same dimensions.

15. The system of claim 11, further comprising a second pouch containing components for carrying out a polymerase chain reaction, wherein the thermal cycler further comprises opposing third and fourth heat-transfer surfaces, and the second pouch is sandwiched between and in contact with the third and fourth heat-transfer surfaces.

16. The system of claim 11, wherein the thermal cycler comprises a first Peltier unit defining the first heat-transfer surface, and a second Peltier unit defining the second heat-transfer surface.

17. The system of claim 11, wherein the thermal cycler comprises a first fan for cooling the first heat-transfer surface and a second fan, separate from the first fan, for cooling the second heat transfer surface.

18. The system of claim 11, further comprising:
a first heat-transfer surface frame stabilizing the first heat-transfer surface;
a second heat-transfer surface frame stabilizing the second heat-transfer surface; and
a linkage linking together the first heat-transfer surface frame and the second heat-transfer surface frame, wherein the linkage is configured to enable at least one of the first heat-transfer surface frame and the second heat-transfer surface frame to be moved toward and away from the other.

19. The system of claim 18, further comprising a cam assembly configured to adjust the distance of a gap formed between the first heat-transfer surface and the second heat-transfer surface when the first heat-transfer surface frame and the second heat-transfer surface frame are operationally positioned for thermal cycling.

20. The system of claim 18, further comprising a housing, wherein the first heat-transfer surface frame is fixed to the housing and the second heat-transfer surface frame is moveable relative to the housing.
